SEQUENCE LISTING (2 sequences)

SEQ ID NO: 1 - DNA, 897 bp, Homo sapiens
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 2 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

(19) United States
(12) Patent Application Publication
(10) Pub. No.: US 2007/0254832 A1
(43) Pub. Date: Nov. 1, 2007

(54) Methods for the treatment of macular degeneration and related eye conditions

(76) Inventors: Milton L. Pressler, Ann Arbor, MI (US); William Sasiela, Montgomery Township, NJ (US); Jorge G. Arroyo, Boston, MA (US); Peter Ganz, Weston, MA (US); Mark A. Creager, Hingham, MA (US)

Correspondence Address: JONES DAY, 222 EAST 41ST ST, NEW YORK, NY, 10017, US

(21) Appl. No.: 11/707,809

(22) Filed: Feb. 15, 2007

Related U.S. Application Data
(60) Provisional application No. 60/774,331, filed on Feb. 17, 2006.
(60) Provisional application No. 60/775,915, filed on Feb. 22, 2006.

(51) Int. Cl. A61K 38/00

(52) U.S. Cl. 514/2

(57) ABSTRACT

The invention provides an AIM-lipid complex for treating age-related macular degeneration and related conditions. The compositions and methods of the instant invention encompass a novel approach to the treatment of age-related macular degeneration and related conditions. Pharmaceutical compositions for this use are also provided.

### 1. FIELD OF THE INVENTION

The present invention is directed to the fields of ophthalmology and cell biology of vision. Specifically, the present invention regards the treatment, amelioration or prevention of age-related macular degeneration (ARMD), including nonexudative (Dry ARMD) and exudative (Wet ARMD) forms. The present invention encompasses novel compositions and methods to treat ARMD and related eye disorders. In one embodiment, the method utilizes, or the composition comprises, an apolipoprotein A-I Milano (AIM) or a apolipoprotein A-I Milano-lipid complex.

### 2. BACKGROUND OF THE INVENTION

2.1 Age-Related Macular Degeneration

Age-related macular degeneration (ARMD) is one of the leading causes of severe visual loss in the developed world (Taylor et al., Br J. Ophtalmol 85:261-266, 2001; VanNewkirk et al., Ophtalmol 107:1593-1600, 2000). In the early stages of the disease, before visual loss occurs from choroidal neovascularization, there is progressive accumulation of lipids in Bruch's membrane (Pauleikhoff et al., PNAS USA 94:4647-4652, 1990; Holz et al., Arch Ophtalmol 112:402-406, 1994; Sheraidah et al., Ophtalmol 100:47-51, 1993; Spaide et al., Retina 19:141-147, 1999). Bruch's membrane lies at the critical juncture between the outer retina and its blood supply, the choriocapillaris. Progressive lipid deposition causes reduced hydraulic conductivity and macromolecular permeability in Bruch's membrane and thereby may impair retinal metabolism (Moore et al., Invenst Optalmol Vis Sci 36:1290-1297, 1995; Pauleikhoffet al., PNAS USA 94:4647-4652,1990; Starita et al., Exp Eye Res 62:565-572, 1996). After sufficient deposition of cholesterol and other lipids in Bruch's membrane, retinal pigmented epithelial cells (RPE) may respond by elaboration of angiogenic factors (e.g. VEGF, vFGF) that promote growth of new vessels from the choroid.

Thus, reduced hydraulic conductivity is one possible explanation for RPE and retina ischemia. The other explanation is the decreased choroidal perfusion, which normally decreases with age and decreases more severely in patient with ARMD. Worsening levels of choroidal perfusion are accosiated with more severe levels of ARMD (Spraul et al., Invest Ophtalmol Vis Sci, 39(11):2201-2202, 1998; Grunwald et al., Invest Ophtalmol Vis Sci, 46(3):1033-1038, 2005; Ciulla et al., Br J Ophtalmol. 86(2):209-213,2002). Also, there is a histologic evidence of choroidal arteriosclerosis (Curcio et al., Invest Ophtalmol Vis Sci, 42:265, 2001).

An open question is the pathogenesis of lipid deposition that ultimately triggers neovascularization. Interestingly, there are parallels between the lipid accumulation in Bruch's membrane found in ARMD and that observed in an animal model of atherosclerosis, the apolipoprotein E (apo E) null mice (Dithmar et al., Invest Ophtalmol Vis Sci., 41:2035-2042, 2000; Kliffen et al., Br J Ophtalmol 84:1415-1419, 2000). Immunohistochemistry on post-mortem eyes has demonstrated apo E in the basal aspect of the retinal pigmented epithelium (RPE) (Anderson et al., Am J Ophtalmol 131)6):767-768, 2001). Cultured RPE cells synthesize high levels of apo E mRNA, comparable to levels found in brain (Anderson et al., 2001, supra). Apolipoprotein E and apo E alleles may be a common denominator associated with several age-related degenerations, for example Alzheimer's disease and atherosclerosis. These associations have stimulated recent investigation of the potential role of apo E in ARMD. Several studies on apo E polymorphism in ARMD have been conducted to find linkages to specific alleles (Simonelli et al., Ophtalmic Res 33:325-328, 2001; Klayer et al., Am J Hum Genet 63(1):200-206, 1998; Souied et al., Am J Ophtalmol 125(3):353-359, 1998). In contrast to Alzheimer's disease, the apo E-4 allele has been associated with reduced prevalence of ARMD. Apo E-2 allele is slightly increased in patients with ARMD. Further supporting a role in ARMD pathogenesis, apo E has been detected in drusens, the Bruch's membrane deposits that are the hallmark of ARMD (Klayer et al., 1998, supra; Anderson et al., 2001, supra).

While the role of apo E in ARMD is suggested but not established, this apolipoprotein has several functions that may affect the course of this disease. Apo E has anti-angiogenic (Browning et al., J Exp Med 180(5): 1949-1954, 1994), anti-inflammatory (Michael et al., in Cellular Immunology, vol. 159, issue 2, pages 124-139, 1994), and anti-oxidative effects (Tangirala et al., E J Biol Chem 276(1): 261-266, 2001). These are all considered atheroprotective attributes of Apo E, but may also be important in protecting against progression of ARMD. While atheroprotective effects of apo E were initially thought to stem from its effects on plasma lipid levels, local effects on vascular macrophages are probably equally important. Thus, selective enhanced expression of macrophage apo E in the arterial wall reduces atherosclerosis in spite of hyperlipidemia (Shimano et al., J Clin Invest 95:469-476, 1995; Bellosta et al., J Clin Invest 96:2170-2177,1995; Hasty et al., Circulation 99:2571-2576, 1999). Conversely, reduction of Apo E levels by reconstitution of apo E null macrophages into C57BL/6 wild type mice fosters the development of atherosclerosis (Fazio et al., 1994).

Atheroprotective effects of arterial apo E expression are thought to derive in part from facilitation of reverse cholesterol transport (Mazzone et al., Circulation 86 (Suppl I): 1-2, 1992; Lin et al., E J Lipid Res 40:1618-1626, 1999). The mechanisms by which apo E facilitates reverse cholesterol transport are incompletely understood. Apo E expression increases cholesterol efflux to HDL3 in J774 macrophages (Mazzone and Reardon, J Lipid Res 35:1345-1353, 1994) and lipid free apolipoprotein Al (Langer et al., J Mol Med 78:217-227, 2000). Cell surface apo E is also hypothesized to induce efflux from the plasma membrane (Lin et al., 1999, supra).

Cholesterol transport may be important in the pathogenesis of ARMD because of lipid efflux from RPE into Bruch's membrane. Very much like intimal macrophages, RPE cells progressively accumulate lipid deposits throughout life; however, unlike vessel wall macrophages, the source of RPE lipid is thought to be retinal photoreceptor outer segments (POS) (Kennedy et al., Eye 9:262-274, 1995). Every day, each RPE cell phagocytoses and degrades more than one thousand POS via lysosomal enzymes. These POS are enriched in phospholipid and contain the photoreactive pigment, rhodopsin. Incompletely digested POS accumulate as lipofuscin in RPE. By age 80, approximately 20% of RPE cell volume is occupied by lipofuscin (Feeney-Burns et al., J Invest Ophtalmol Vis Sci 25:195-200, 1984).

Analysis of Bruch's membrane lipid reveals an age-related accumulation of phospholipid, triglyceride, cholesterol, and cholesterol ester (Holz et al., 1994, supra; Curcio et al., J Invest Ophtalmol Vis Sci 42:265, 2001). The origin of these lipids also is thought to derive principally from POS rather than from the circulation (Holz et al., 1994, supra; Spaide et al., 1999, supra). POS lipids are hypothesized to efflux from the RPE into Bruch's membrane. Although cholesterol ester deposition in Bruch's suggests contribution from plasma lipids, biochemical analysis of these ethers suggests etherification of intracellular cholesterol by RPE cell derived ACAT (Curcio et al., ARVO Abstracts, 2002). While trafficking of lipids from the retina to RPE cells has been studied extensively, mechanisms of lipid efflux from RPE to Bruch's membrane are not well understood. Furthermore, from a pathogenic standpoint, regulation of lipid efflux into Bruch's membrane may be important in determining the rate of lipid-induced thickening that occurs in aging.

The three possible sources for cholesterol in Bruch's membrane include cellular cholesterol and plasma lipids described above, as well as an in situ synthesis. The brain, for example, synthesizes its own cholesterol, obtains very little of it from the plasma, and has a very slow cholesterol turn over.

Reverse cholesterol transport in macrophages is regulated by nuclear hormone receptor ligands via their effects on ABCA-1 and apo E expression. Liver X receptor (LXR) and/or retinoid X receptor (RXR) ligands increase levels of these transporters and increase reverse cholesterol transport in macrophages (Mak et al., J Biol Chem 277(35):31900-31908, 2002; Laffitte et al., PNAS USA 98(2):507-512, 2001). Thyroid hormone has also been demonstrated to increase expression of apo E three fold in HepG2 cells (Laffitte et al., Eur J Biochem 224(2):463-471, 1994).

In atherosclerosis (AS), lipids accumulate in the extracellular matrix and within phagocytic cells, primarily macrophages. Mechanisms of lipid metabolism in AS have been investigated in detail. Similar investigations into lipid processing by RPE and subsequent lipid efflux into BM and the circulation have not been conducted with the same depth as those for AS. As a consequence, potential therapeutic approaches to dry ARMD are wanting.

Mullins (Mullins et al., FASEB J 14(7):835-846, 2000) describes compositional similarity between drusen and other extracellular deposits, including atherosclerotic plaques. Specifically, vitronectin, amyloid P, Apo E and lipids are among the constituents shared in common. More specifically, apolipoprotein E is identified in retinal pigmented epithelium.

Friedman (Friedman, Am J Ophtalmol 130(5):658-663, 2000) reviews the role of atherosclerosis in the pathogenesis of ARMD. Specifically, the review mentions targeting the angiogenesis pathway for treating the neovascular form of ARMD, such as the member VEGF. It is noted that interfering with the upregulation or action of angiogenic agents may prove helpful for choroidal neovascularization, and, in alternative embodiments, statins may be useful for lowering the risk of ARMD.

Anderson et al. (Anderson et al., 2001, supra) reports that apolipoprotein E protein is found in the same location as drusen, likely originating from the retinal pigmented epithelium.

U.S. Pat. No. 6,071,924 regards inhibition of proliferation of retinal pigmented epithelium by contacting RPE cells with a retinoic acid receptor agonist, except for retinoic acid, preferably thereby inhibiting AP-1-dependent gene expression. In specific embodiments, an AP1 antagonist is delivered to a subject in need thereof for inhibition of proliferation of retinal pigment epithelium or a disease associated therewith.

U.S. Pat. No. 6,075,032 is directed to inhibition of choroidal neovascularization by contacting RPE cells with an AP-1 antagonist.

U.S. Pat. No. 5,824,685 regards amelioration of proliferative vitreoretinopathy or traction retinal detachment by contacting RPE cells with a retinoic acid receptor selected from ethyl-6-[2-(4,4-dimethylthiochroman-6-yl)ethynyl]nicotinate, 6-[2-(4,4-dimethylchroman-6-yl) ethynyl]nicotinic acid, and p-[(E)-2-(5,6,7,8-tetrahydro-5,5,8,8-tetramethyl-2-naphthyl)propenyl]-benzoic acid.

U.S. Pat. No. 6,372,753 addresses inhibition of an ocular disease resulting from proliferation of retinal pigmented epithelium by providing at least one AP-1 antagonist and at least one retinoic acid receptor (RAR) agonist, except for retinoic acid.

WO 01/58494 is directed to treating or preventing an ocular disease, such as age-related macular degeneration, by contacting an ocular cell with an expression vector comprising a nucleic acid sequence encoding an inhibitor of angiogenesis and a neurotrophic agent. In specific embodiments, the inhibitor of angiogenesis and the neurotrophic agent are one and the same, such as pigment epithelium-derived factor (PEDF).

WO 02/13812 regards the use of an insulin-sensitizing agent, preferably peroxisome proliferator-activated receptor y (PPAR y) agonists, for the treatment of an inflammatory disease, such as an ophthalmic disease.

WO 00/52479 addresses diagnosing, treating, and preventing drusen-associated disorders (any disorder which involves drusen formation), including ARMD. In specific embodiments, there are methods related to providing an effective amount of an agent that inhibits immune cell proliferation or differentiation, such as antagonists of the cytokine, tumor necrosis factor (TNF)-alpha.

WO 2004/098506 and WO 2004/0266663 describe the treatment of age-related macular degeneration using regulation of pathogenic mechanisms similar to atherosclerosis. In specific embodiments, reverse cholesterol transport components, such as transporters and HDL fractions, are utilized as diagnostic and therapeutic targets for age-related macular degeneration. In a further specific embodiment, the lipid content of the retinal pigmented epithelium, and/or Bruch's membrane is reduced by delivering Apolipoprotein Al, particularly a mimetic peptide.

Arroyo in the “Age-related macular degeneration-I and II,” UpToDate (on-line service) (last modified Sep. 19, 2005), provides background information on age-related macular degeneration, including epidemiology, etiology/risk factors, clinical presentation, diagnosis, treatment and prevention.

Duncan et al. (Duncan et al., Br J Ophthalmol 89(12):1549-51, 2005) describes that the findings in LDL receptor deficient mice may provide insight into the mechanism of early ARMD.

Rudolf et al. (Rudolf et al., 2005) describes that LDL receptor deficient mice exhibit an accumulation of lipid particles in Bruch's membrane which is further increased after fat intake.

Nissen et al. (Nissen et al., JAMA 290:2292-2300, 2003) discloses a study in which atheroma volume is reduced after treatment with recombinant apoA-I Milano-POPC complex.

2.2 Apolipoprotein A-I Milano

Human Apo-I Milano is a natural variant of the wild-type apolipoprotein, Apo A-I (Weisgraber et al., J Clin Invest 66:901-907, 1980). In Apo A-I Milano the amino acid arginine (Arg173) is replaced by the amino acid cysteine (Cys173). Subjects with Apo A-I Milano are characterized by having low levels of plasma HDL cholesterol and Apo A-I. See, for example, Parolini(Parolini et al, Atherosclerosis 183(2):222-229, 2005), Chiesa and Sirtori (Chiesa and Sirtori, Curr Opin Lipidol 14(2):159-163, 2003) and Genbank Accession No. NP-000030.1 (gi 4557321).

The presence of one cysteine residue per Apo A-I Milano molecule enables the formation of disulfide bonds between like or unlike polypeptide chains. Hence, within lipoprotein particles Apo A-I Milano may exist in a monomeric, homodimeric, or heterodimeric forms (See, e.g., U.S. Pat. No. 5,876,968). These forms are chemically interchangeable, and the term Apo A-I Milano does not discriminate between these forms. On the DNA level the variant form results from a C to T substitution in the gene sequence, i.e., the codon CGC changed to TGC, allowing the translation of a cysteine (Cys) instead of arginine (Arg) at amino acid position 173.

Methods for obtaining Apo A-I Milano are well-known in the art. For example, Apo A-I Milano can be separated from plasma, for example, by density gradient centrifugation followed by delipidation of the lipoprotein, reduction, denaturization and gel-filtration chromatography, ion-exchange chromatography, hydrophobic, e.g., phenyl sepharose, interaction chromatography or immunoaffinity chromatography, or produced synthetically, semi-synthetically, or using recombinant DNA techniques and subsequent purification techniques known to those skilled in the art. (See, e.g., U.S. Pat. Nos. 6,107,467; 6,559,284; 6,423,830; 6,090,921; 5,834,596; 5,990,081; 6,506,879; 5,059528; 5,876,968; and 5,721,114; Mulugeta et al., J. Chromatogr 798(1-2): 83-90 (1998); Chung et al., J. Lipid Res 21(3):284-91 (1980); Cheung et al., J. Lipid Res 28(8):913-29 (1987); Persson et al. J. Chromatogr 711:97-109 (1998); and PCT Publications WO 86/04920 and WO 87/02062).

Thus, there is a need for a new approach for the treatment, amelioration or prevention of age-related macular degeneration by reducing the size and lipid content of drusens and other pathological lesions of the retina and ocular tissue, such as Bruch's membrane.

### 3. SUMMARY OF THE INVENTION

The invention encompasses methods and compositions for the treatment of age-related macular degeneration or a related disorder in a mammal using exogenously produced lipoprotein particles acting as an HDL mimetic, such as an Apo A-I Milano or an AIM:phospholipid complex.

Methods, compositions and dosage regimens are provided herein, and are believed to encompass safe, effective and non-surgical treatments, without being limited by theory, that rapidly promote cholesterol efflux and mobilization from lipidic plaques in the retina, RPE or Bruch's membrane (e.g. drusens), which thereby confer benefit in terms of improvement of visual acuity, prevention of loss of visual acuity, or improvement or prevention of macular degeneration that leads to impaired visual acuity. The mechanism of action encompasses improved blood flow or perfusion of the retina or choroid plexus that directly or indirectly confers improvement in visual acuity, or prevention of loss of visual acuity, or confers amelioration of lesions that promote neovascularization of the retina that reduce visual acuity.

The methods of the invention comprise administering to a mammal in need thereof an Apo A-I Milano (“AIM”) protein in soluble or particulate form or an AIM:lipid complex. In the methods of the present invention an Apo A-I Milano can be complexed with a lipid or phospholipid. The invention encompasses treatment with Apo A-I Milano particles containing any suitable phospholipid, in one embodiment the phospholipid is phospholipid POPC, that are co-administered with the variant apolipoprotein in a typical therapeutic particle. As used herein, Apo A-I Milano, or lipid or phospholipid complexes thereof, may or may not be in the form of a pharmaceutical composition.

The pharmaceutical compositions of the present invention may comprise a single active ingredient selected from one or more Apo A-I Milano or derivatives thereof and one or more peptide:lipid complexes such as AIM:lipid complex. The Apo A-I Milano can be any recombinant, synthetic or purified human or non-human Apo A-I Milano obtained from any source available by any method well-known in the art. In one embodiment, the Apo A-I Milano is a recombinant protein. In another embodiment, the ApoA-I Milano is a human ApoA-I Milano. In the methods and compositions of the present invention, an Apo A-I Milano can be administered to a mammal in need thereof in a dose of about 1 mg/kg to about 50 mg/kg.

The present invention provides pharmaceutical kits which comprise a dosage form of Apo A-I Milano or a dosage form of Apo A-I Milano complexed with a lipid or phospholipid in a particulate or emulsion form. The pharmaceutical formulations can be labeled and have accompanying labeling to identify the formulation contained therein and other information useful to health care providers and subjects in the prevention or treatment of age-related macular degeneration, including, but not limited to, instructions for use, dose, dosing interval, duration, indication, contradictions, warnings, precautions, handling and storage instructions and the like.

### 4. DETAILED DESCRIPTION OF THE INVENTION

4.1 Definitions

As used herein, the following terms shall have the following meaning:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “age-related macular degeneration” as used herein refers to macular degeneration, both wet and dry forms, early or late stage, usually in an individual over the age of about 50, but also in rare juvenile forms of the disease. In one specific embodiment, it is associated with destruction and loss of the photoreceptors in the macula region of the retina resulting in decreased central vision and, in advanced cases, legal blindness.

The term “Bruch's membrane” as used herein refers to a five-layered structure separating the choriocapillaris from the retinal pigmented epithelium, RPE. This structure is composed of the basal lamina of the RPE and choriocapillaris, two collagen layers and one elastic layer.

The term “increase lipid efflux” or “increasing lipid efflux” as used herein refers to an increased level and/or rate of lipid efflux, promoting lipid efflux, enhancing lipid efflux, facilitating lipid efflux, upregulating lipid efflux, improving lipid efflux, and/or augmenting lipid efflux. In one embodiment, the efflux comprises efflux of phospholipid, triglyceride, cholesterol, and/or cholesterol ester.

The term “reverse cholesterol transport” refers to the net movement (e.g., efflux or transport) of cholesterol, for example, cholesterol synthesized in extrahepatic tissues or acquired from lipoproteins, to the liver for excretion in the bile.

The term “macula” as used herein refers to the light-sensing cells or photoreceptors of the central region of the retina.

The term “macular degeneration” as used herein refers to deterioration of the central portion of the retina, the macula.

The terms “treat”, “treating” or “treatment” refer to alleviating, reducing, abrogating, or otherwise modulating a disease, disorder and/or one or more symptoms thereof, that is a therapeutic effect on an existing condition

The term “therapeutically effective amount” refers to that amount of an active ingredient sufficient to improve one or more of the symptoms of the condition or disorder being treated as compared to those symptoms that occur without treatment. The improvement may be temporary or permanent.

The term “prophylactically effective amount” refers to that amount of an active ingredient sufficient to result in the prevention, onset or recurrence of one or more symptoms of the condition or disorder.

The terms “prevent”, “preventing” or “prevention” refer to barring, or reducing the risk of, a subject from acquiring a disease, disorder and/or symptoms thereof.

The term “pharmaceutical formulation” refers to a composition comprising either an active ingredient and a suitable diluent, carrier, vehicle, or excipients suitable for administration to a subject. The pharmaceutical formulation or composition will comprise ApoA-I Milano. The terms “composition” and “formulation” are used interchangeably herein. The term is also meant to encompass situations wherein the components of the combination therapy are in the same or separate formulations. This term includes, but is not limited to oral, parenteral, intraocular, periocular, mucosal and topical compositions as described below.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

The term “label” refers to a display of written, printed or graphic matter upon the immediate container of an article, for example, the written material displayed on a vial containing a pharmaceutically active agent.

The term “labeling” refers to all labels and other written, printed or graphic matter upon any article or any of its containers or wrappers or accompanying such article, for example, a package insert, instructional videotapes or instructional DVDs accompanying or associated with a container of a pharmaceutically active agent.

4.2 Pharmaceutical Compositions of the Invention

The pharmaceutical compositions of the present invention comprise an active ingredient selected from one or more ApoA-I Milano or a derivative thereof and one or more peptide:lipid complexes such as AIM:lipid complex.

The pharmaceutical compositions of the present invention comprise pharmaceutically acceptable excipients as required to approximate physiological conditions, such as pH adjusting agents, buffering agents, and tonicity adjusting agents (e.g., sodium acetate, sodium lactate, sodium chloride, potassium chloride, and calcium chloride). They may also comprise osmotically active cryoprotectants like mannitol or sucrose. Antibacterial agents (e.g., phenol, benzalkonium chloride or benzethonium chloride) can be added to maintain sterility of a product, especially to pharmaceutical formulations intended for multi-dose parenteral use. Suspending, stabilizing and/or dispersing agents can also be used in the compositions of the invention.

The pharmaceutical formulations can be in a variety of forms suitable for any route of administration which include, but are not limited to parenteral, intraocular, periocular, subcutaneous, oral, transdermal, transmucosal and rectal administration.

In one embodiment, the composition is formulated in accordance with routine procedures as a pharmaceutical composition adapted for parental administration.

Parenteral administration refers to any route of administration that is not through the alimentary canal, including, but not limited to, injectable administration (i.e., intravenous, intraarterial, intramuscular, intradermal, intraocular, periocular and the like as described herein) (see generally, Remington's Pharmaceutical Sciences, 18^{th} Edition, Gennaro et al., eds., Mack Printing Company, Easton, Pa., 1990).

The pharmaceutical formulation of the present invention can be in a form suitable for an ocular route of administration, which includes, but is not limited to an intravitreal, intraocular (intracameral), subconjunctival, sub-Tenon's, retrobulbar injection and a topical application. In one embodiment, the pharmaceutical formulation is administered as an intravitreal injection. The injectable pharmaceutical formulation of the present invention is ophtalmically acceptable, i.e., it is appropriate for administration directly into the eye including aqueous and vitreous humors.

Injectable pharmaceutical formulations can be sterile suspensions, solutions or emulsions in aqueous or oily vehicles. The formulations for injection can be presented in unit dosage form, e.g., in ampules or in multidose containers, and can comprise added preservatives. In one embodiment, the buffers for parenteral pharmaceutical formulations are phosphate, citrate and acetate, and may contain stabilizers or cryopreservatives (e.g., sucrose, mannitol, trehalose).

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms of the invention.

The pharmaceutical formulations can be formulated for a single, one-time use or can be formulated in multiple doses. The components of the combination therapy can be in the same or different pharmaceutical formulations and can be administered simultaneously or sequentially. In certain embodiments, the pharmaceutical formulation can be lyophilized in sterile bottles or in sterile pre-filled syringes or sterile pre-filled bags which can be frozen or refrigerated. The formulation can also be lyophilized and reconstituted with a suitable vehicle.

4.3 Use of Apolipoprotein A-I Milano

In one aspect, the present invention provides compositions and methods for the treatment of age-related macular degeneration, its various forms (e.g., wet, dry; early, late) or related conditions by administering a composition comprising normal human Apo A-I, Apo A-I Milano, or other biologically active variants thereof. The human Apo A-I nucleotide sequence is set forth in SEQ ID NO: 1 shown below. The coding sequence includes nucleotides 39-842.

(SEQ ID NO:1)

AGAGACTGCGAGAAGGAGGTCCCCCACGGCCCTTCAGGATGAAAGCTGCG
GTGCTGACCTTGGCCGTGCTCTTCCTGACGGGGAGCCAGGCTCGGCATTT
CTGGCAGCAAGATGAACCCCCCCAGAGCCCCTGGGATCGAGTGAAGGACC
TGGCCACTGTGTACGTGGATGTGCTCAAAGACAGCGGCAGAGACTATGTG
TCCCAGTTTGAAGGCTCCGCCTTGGGAAAACAGCTAAACCTAAAGCTCCT
TGACAACTGGGACAGCGTGACCTCCACCTTCAGCAAGCTGCGCGAACAGC
TCGGCCCTGTGACCCAGGAGTTCTGGGATAACCTGGAAAAGGAGACAGAG
GGCCTGAGGCAGGAGATGAGCAAGGATCTGGAGGAGGTGAAGGCCAAGGT
GCAGCCCTACCTGGACGACTTCCAGAAGAAGTGGCAGGAGGAGATGGAGC
TCTACCGCCAGAAGGTGGAGCCGCTGCGCGCAGAGCTCCAAGAGGGCGCG
CGCCAGAAGCTGCACGAGCTGCAAGAGAAGCTGAGCCCACTGGGCGAGGA
GATGCGCGACCGCGCGCGCGCCCATGTGGACGCGCTGCGCACGCATCTGG
CCCCCTACAGCGACGAGCTGCGCCAGCGCTTGGCCGCGCGCCTTGAGGCT
CTCAAGGAGAACGGCGGCGCCAGACTGGCCGAGTACCACGCCAAGGCCAC
CGAGCATCTGAGCACGCTCAGCGAGAAGGCCAAGCCCGCGCTCGAGGACC
TCCGCCAAGGCCTGCTGCCCGTGCTGGAGAGCTTCAAGCTCAGCTTCCTG
AGCGCTCTCGAGGAGTACACTAAGAAGCTCAACACCCAGTGAGGCGCCCG
CCGCCGCCCCCCTTCCCGGTGCTCAGAATAAACGTTTCCAAAGTGGG

This gene encodes Apo A-I, which is the major protein component of high density lipoprotein (HDL) in plasma. The protein promotes cholesterol efflux from tissues to the liver for excretion, and it is a co-factor for lecithin cholesterolacyltransferase (LCAT), which is responsible for the formation of most plasma cholesteryl esters (see, Law and Brewer, PNAS USA 81(1):66-70, 1984; Genbank Accession No. NM-000039.1 (gi 4557320). The human Apo A-I preprotein amino acid sequence is set forth in SEQ ID NO:2 shown below.

(SEQ ID NO:2)

MKAAVLTLAVLFLTGSQARHFWQQDEPPQSPWDRVKDLATVYVDVLKDSG
RDYVSQFEGSALGKQLNLKLLDNWDSVTSTFSKLREQLGPVTQEFWDNLE
KETEGLRQEMSKDLEEVKAKVQPYLDDFQKKWQEEMELYRQKVEPLRAEL
QEGARQKLHELQEKLSPLGEEMRDRARAHVDALRTHLAPYSDELRQRLAA
RLEALKENGGARLAEYHAKATEHLSTLSEKAKPALEDLRQGLLPVLESFK
VSFLSALEEYTKKLNTQ

In one embodiment, the present invention provides compositions and methods for the treatment age-related macular degeneration, its various forms (e.g., wet, dry; early, late) or related conditions by administering a composition comprising Apo A-I Milano to a subject in need thereof. In certain embodiments the Apo A-I Milano can be complexed with a lipid or phospholipid. As used herein, Apo A-I Milano, or lipid complexes thereof, may or may not be in the form of a pharmaceutical formulation.

In certain embodiments, the Apo A-I Milano can be a variant of or conservatively substituted Apo A-I Milano. By “conservative substitution,” it is meant that certain amino acid residues of Apo A-I Milano can be replaced with other amino acid residues without significantly deleteriously affecting the activity of the protein. For example, Apo A-I proteins having various cysteine substitutions (e.g., one or more cysteine substitutions distinct from that of the Apo A-I mutant) throughout the protein could achieve the desired functional result by promoting dimer formation in a manner similar to that seen for the Apo A-I mutant. Thus, also contemplated by the present invention are altered or substituted forms of Apo A-I Milano, wherein at least one defined amino acid residue in the structure is substituted with another amino acid residue.

The Apo A-I Milano utilized in the invention can be obtained from any source available. For example, the Apo A-I Milano can be recombinant, synthetic, semi-synthetic or purified Apo A-I Milano. In one embodiment, the Apo A-I Milano is a recombinant protein (rApo A-I Milano or rAIM) expressed in yeast or E. coli as described in U.S. Pat. No. 5,721,114 and European Patents EP 0 469 017 and EP 0 267 703.

Methods for obtaining Apo A-I Milano utilized by the invention are well-known in the art. For example, Apo A-I Milano can be separated from plasma, for example, by density gradient centrifugation followed by delipidation of the lipoprotein, reduction, denaturazation and gel-filtration chromatography, ion-exchanging chromatography, hydrophobic, e.g., phenyl sepharose, interaction chromatography or immunoaffinity chromatography, or produced synthetically, semi-synthetically or using recombinant DNA techniques known to those skilled in the art and subsequent purification familiar to those skilled in the art (See, e.g., U.S. Pat. Nos. 6,107,467; 6,559,284; 6,423,830; 6,090,921; 5,834,596; 5,990,081; 6,506,879; 5,059,528; 5,876,968 and 5,721,114; Mulugeta et al., J. Chromatogr 798(1-2): 83-90 (1998); Chung et al., J. Lipid Res 21(3):284-91 (1980); Cheung et al., J. Lipid Res 28(8):913-29 (1987); Persson et al., J. Chromatogr 711:97-109 (1998); and PCT Publications WO 86/04920 and WO 87/02062).

If the Apo A-I Milano is obtained from natural sources, it can be obtained from any animal source of any species. In certain embodiments, the Apo A-I Milano is obtained from a mammalian source. In certain embodiments, the Apo A-I Milano is obtained from a human source. In a specific embodiment of the invention, the Apo A-I Milano is derived from the same species as the mammal to which the Apo A-I Milano is administered.

4.3.1 Expression Of Genes Encoding Apo A-I Milano

The nucleotide sequence coding for a A-I Milano or variants thereof can be inserted into an appropriate expression vector, i.e., a vector which contains the necessary elements for the transcription and translation of the inserted protein-coding sequence. The necessary transcriptional and translational signals can also be supplied by the native A-I Milano gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of nucleic acid sequence encoding a A-I Milano may be regulated by a second nucleic acid sequence so that the A-I Milano is expressed in a host transformed with the recombinant DNA molecule. For example, expression of A-I Milano may be controlled by any promoter/enhancer element known in the art. In a specific embodiment, the promoter is heterologous to (i.e., not a native promoter of) the specific A-I Milano-encoding gene. Promoters that may be used to control expression of A-I Milano-encoding genes include, but are not limited to, the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), the regulatory sequences of the metallothionein gene (Brinster et al., Nature 296:39-42 (1982)); prokaryotic expression vectors such as the ∃-lactamase promoter (Villa-Kamaroff et al., Proc. Natl. Acad. Sci. U.S.A. 75:3727-3731 (1978)), or the tat promoter (DeBoer et al., Proc. Natl. Acad. Sci. U.S.A. 80:21-25 (1983)).

A person of skill in the art will appreciate that cDNA, genomic, and synthesized sequences can be cloned and expressed. One way to accomplish such expression is by transferring an A-I Milano gene, or a nucleic acid encoding an A-I Milano or variant thereof, to cells in tissue culture. The expression of the transferred gene may be controlled by its native promoter, or can be controlled by a non-native promoter. In addition to transferring a nucleic acid comprising a nucleic acid sequence encoding an A-I Milano, the transferred nucleic acids can encode a functional portion of a particular A-I Milano, or a protein having at least 60% sequence identity to an A-I Milano disclosed herein, as compared over the length of the particular A-I Milano, or a polypeptide having at least 60% sequence similarity to an A-I Milano variant, as compared over the length of the A-I Milano variant. Introduction of the nucleic acid into the cell is accomplished by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene.

4.3.2 Lipid Complexes of Apolipoprotein A-I Milano

In certain embodiments, the compositions of the invention comprise lipid complexes of Apo A-I Milano. In some embodiments, the invention provides pharmaceutical formulations of AIM:phospholipid complexes. Efficacy can be enhanced by the complexing of lipids or phospholipids with Apo A-I Milano. Typically, the lipid or phospholipid is mixed with the Apo A-I Milano prior to administration. Apo A-I Milano and lipids can be mixed in an aqueous solution in appropriate ratios and can be complexed by methods known in the art including freeze-drying or freeze-thaw cycles, detergent solubilization followed by dialysis, microfluidization, sonication, and homogenization. Complex efficiency can be optimized, for example, by varying pressure, ultrasonic frequency, or detergent concentration. An example of a detergent commonly used to prepare AIM:phospholipid complexes is sodium cholate.

In some cases, the Apo A-I Milano is administered alone, essentially lipid-free, to treat age-related macular degeneration including its various forms. In other embodiments, therapy comprising an AIM:lipid complex is administered to a subject in need thereof.

In one embodiment, the AIM:phospholipid complex is in solution with an appropriate pharmaceutical diluent. In another embodiment, freeze-dried or lyophilized preparations of AIM:phospholipid complexes can be hydrated or reconstituted with an appropriate pharmaceutical diluent prior to administration. In yet another embodiment, the AIM:lipid complexes can be frozen preparations that are thawed until a homogenous solution is achieved prior to administration to a subject in need thereof.

The lipid can be any suitable lipid known to those of skill in the art. Non-phosphorus containing lipids can be used, including stearylamine, dodecylamine, acetyl palmitate, (1,3)-D-mannosyl-(1,3)diglyceride, aminophenylglycoside, 3-cholesteryl-6′-(glycosylthio)hexyl ether glycolipids, N-(2,3-di(9-(Z)-octadecenyloxy))-prop-1-yl-N,N,N-trimethylammonium chloride and fatty acid amides.

In one embodiment, the lipid is a phospholipid. The phospholipid can be obtained from any source known to those of skill in the art. For example, the phospholipid can be obtained from commercial sources, natural sources or by synthetic or semi-synthetic means known to those of skill in the art (see, e.g., Mel'nichuk et al., Ukr Biokhim Zh 59(6):75-7 (1987); Mel'nichuk et al., Ukr Biokhim Zh 59(5):66-70 (1987); Ramesh et al., J Am Oil Chem Soc 56(5):585-7 (1979); Patel and Sparrow, J. Chromatogr 150(2):542-7 (1978); Kaduce et al., J. Lipid Res 24(10):1398-403 (1983); Schlueter et al., Org. Lett 5(3):255-7 (2003); Tsuji et al., Nippon Yakurigaku Zasshi 120(1):67P-69P (2002)).

The phospholipid can be any phospholipid known to those of skill in the art. For example, the phospholipid can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives.

The phospholipid can also be a derivative or analogue of any of the above phospholipids. In certain embodiments, the AIM:phospholipid complex comprises combinations of two or more phospholipids.

In one embodiment, the therapy comprises an AIM:phospholipid complex. In another embodiment, the lipid is a phospholipid, for example, 1-palmitoyl-2-oleoyl phosphatidylcholine (“POPC”) or (“1-palmitoyl-2-oleoyl-sn-glycero-3-phosphocholine”). In another embodiment, the AIM:POPC complex comprises about a 1:1 ratio by weight of AIM:POPC. In yet another embodiment, the AIM:POPC complex is a pharmaceutical formulation. The AIM:POPC complex is referred to as ETC-000216 or ETC-216 (Nissen et al., JAMA 290:2292-2300, 2003; also TV Broadcast, “Good Morning America”, Apr. 15, 2004).

The complex comprising Apo A-I Milano and a lipid can comprise any amount of lipid, including phospholipid, and any amount of Apo A-I Milano effective to treat age-related macular degeneration and its various forms. In certain embodiments, the Apo A-I Milano comprises a complex of Apo A-I Milano and a phospholipid in a ratio of about 1:1 by weight. The Apo A-I Milano can comprise complexes with other ratios of phospholipid to Apo A-I Milano, such as about 100:1, about 10:1, about 5:1, about 3:1, about 2:1, about 1:1, about 1:2, about 1:3, about 1:5, about 1:10 or about 1:100. In certain embodiments, a ratio by weight is of between about 1:0.5 to about 1:3, in one embodiment, a ratio is of about 1:0.8 to about 1:1.2 produces the most homogenous population of lipoprotein particles and to produce stable and reproducible batches. In another embodiment, the ratio by weight of Apo A-I Milano to phospholipid is 1:0.95.

Additional lipids suitable for use in the compositions and methods of the invention are well known to persons of skill in the art and are cited in a variety of well known sources, e.g., McCutcheon's Detergents and Emulsifiers and McCutcheon's Functional Materials, Allured Publishing Co., Ridgewood, N. J., both of which are incorporated herein by reference. In certain embodiments, the lipids are liquid-crystalline at 37° C., 35° C., or 32° C. In another embodiment, the lipids that are liquid-crystalline at 37° C.

In certain embodiments, the therapy comprises a concentration of ETC-216 sufficient to treat an age-related macular degeneration or related eye disorder in a mammal in need thereof. In certain embodiments, the concentration of ETC-216 is about 2.5 mg/kg to about 50 mg/kg. In other embodiments, the ETC-216 concentration is about 10 mg/kg to about 20 mg/kg. In another embodiment, the concentration of ETC-216 is about 13 mg/kg to about 16 mg/kg. The concentration of ETC-2 16 can be determined by any suitable technique known to those of skill in the art. In certain embodiments, the ETC-216 concentration is determined by size exclusion high performance liquid chromatography (SE-HPLC).

In certain embodiments, the pharmaceutical formulation comprises sucrose in an amount sufficient to make a pharmaceutically suitable formulation with ETC-216. In certain embodiments, the pharmaceutical formulation comprises about 0.5% to about 20% sucrose. In certain embodiments, the pharmaceutical formulation comprises about 3% to about 12% sucrose. In other embodiments, the pharmaceutical formulation comprises about 5% to about 7% sucrose. In other embodiments, the pharmaceutical formulation comprises about 6.0% to about 6.4% sucrose. In another embodiment, the pharmaceutical formulation comprises 6.2% sucrose.

In certain embodiments, the pharmaceutical formulation comprises mannitol in an amount sufficient to make a pharmaceutically suitable formulation of ETC-216. In some embodiments, the pharmaceutical formulation comprises about 0.01% to about 5% mannitol. In other embodiments, the pharmaceutical formulation comprises about 0.1% to about 3% mannitol. In certain embodiments, the pharmaceutical formulation comprises about 0.5% to about 2% mannitol. In other embodiments, the pharmaceutical formulation comprises about 0.7% to about 1% mannitol. In another embodiment, the pharmaceutical formulation comprises 0.8% mannitol.

In certain embodiments, the pharmaceutical formulation comprises a buffer in an amount sufficient to make a pharmaceutically suitable formulation of Apo A-I Milano or AIM:lipid complex. In some embodiments, the pharmaceutical formulation comprises a phosphate buffer. In particular embodiments, the buffer concentration is about 3 mM to about 25 mM. In other embodiments, the buffer concentration is about 5 mM to about 20 mM. In another embodiments, the buffer concentration is about 8 mM to about 15 mM.

In certain embodiments, an appropriate buffer is added to adjust the pH of the pharmaceutical formulation to a range suitable for administration to a subject. In certain embodiments, the pharmaceutical formulation has a pH of about 6.8 to about 7.8. In some embodiments, the pharmaceutical formulation has a pH of about 7.0 to about 7.8. In other embodiments, the pharmaceutical formulation can have a pH of about 7.2 to about 7.5. In a preferred embodiment, the pharmaceutical formulation has a pH of about 7.4.

4.3.3 Preparation of Lipid Complexes of Apolipoprotein A-I Milano

The AIM:lipid complexes can be made by any method known to one of skill in the art. In some cases, the lipid and Apo A-I Milano are mixed prior to administration. Lipids can be in a solution or in any other form suitable for the preparation of a complex with an Apo A-I Milano.

An AIM:lipid complex can be prepared in a variety of forms, including, but not limited to vesicles, liposomes or proteoliposomes, using methods well known in the art.

In certain embodiments, Apo A-I Milano can be combined with preformed lipid vesicles resulting in the spontaneous formation of an AIM:lipid complex. In another embodiment, the Apo A-I Milano can be made by a detergent dialysis method. For example, a mixture of Apo

A-I Milano, lipid and a detergent, such as cholate, can be dialyzed to remove the detergent and reconstituted to make the lipid complexes. (See, e.g., Jonas et al., Methods Enzymol 128, 553-82 (1986)). In another embodiment, the lipid complexes can be made by co-lyophilization. (See, e.g., U.S. Pat. Nos. 6,004,925, 6,037,323, 6,046,166, 6,287,590 and 6,455,088). Other methods of preparing AIM:lipid complexes will be apparent to those of skill in the art.

In another embodiment, the lipid complexes are made by homogenization. In one embodiment, the AIM:lipid complexes are made by first diluting recombinant Apo A-I Milano to a concentration of 15 mg/ml in solution with water for injection. Sodium phosphate is then added to a final concentration of 9-15 mM to adjust the pH to between about 7.0 and about 7.8. Next, mannitol is added to a concentration of about 0.8% to about 1% (w/v), and POPC is added to achieve a mixture of about 1:0.95 (wt protein/wt lipid). The mixture is stirred at 5000 rpm for about 20 minutes using an overhead propeller and an ULTRA TURRAX dispensing instrument while maintaining the temperature between 37° C. to 43° C. The feed vessel is stirred continuously at 100-300 rpm while the temperature is maintained between 32° C. to 43° C. with in-line heat exchangers (Avestin, Inc.). Homogenization for the first 30 minutes is carried out at 50 MPa (7250 psi) and thereafter, the pressure is maintained at 80-120 MPa (11600-17400 psi) until in-process testing by gel permeation chromatography demonstrates the % AUC of >70% between protein standards.

4.3.4 Pharmaceutical Compositions of ETC-216

The pharmaceutical composition of ETC-216 comprises an Apo A-I Milano:lipid complex suitable for treatment of age-related macular degeneration or related conditions.

In one embodiment, the pharmaceutical composition of Apo A-I Milano:lipid complex comprises a pharmaceutically acceptable carrier or vehicle. Many pharmaceutically acceptable carriers or vehicles can be employed, such as sucrose-mannitol, normal saline, glucose, trehalose, sucrose, sterile water, buffered water, 0.45% saline (half normal saline), and 0.3% glycine, and may further comprise glycoproteins for enhanced stability, such as albumin. These formulations can be sterilized by conventional, well known sterilization techniques. The resulting aqueous solutions can be packaged for use or filtered under aseptic conditions and lyophilized (freeze-dried). The lyophilized preparation can then be combined with a sterile aqueous solution prior to administration.

The pharmaceutical formulations comprising the ETC-216 can be in a salt form. For example, because proteins can comprise acidic and/or basic termini and/or side chains, the ETC-216 can be in the pharmaceutical formulation containing as either a free acid or base forms, or as a pharmaceutically acceptable salt. Suitable acids capable of forming pharmaceutically acceptable salts with the ETC-216 include, for example, inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, flumaric acid, anthranilic acid, cinnamic acid, naphthalene sulfonic acid, sulfanilic acid and the like. Suitable bases capable of forming salts with Apo A-I Milano can include, for example, inorganic bases such as sodium hydroxide, ammonium hydroxide, potassium hydroxide and the like; and organic bases such as mono-, di-and tri-alkyl amines (e.g., triethyl amine, diisopropyl amine, methyl amine, dimethyl amine and the like) and optionally substituted ethanolamines (e.g., ethanolamine, diethanolamine and the like).

In another embodiment, the pharmaceutical formulations can be provided in a powder form or lyophilized form for reconstitution before use with a suitable vehicle, including but not limited to sterile pyrogen free water, saline or dextrose. To this end, the ETC-216 can be lyophilized. In another embodiment, the pharmaceutical formulations can be supplied in unit dosage forms and reconstituted prior to use.

For prolonged delivery, the pharmaceutical formulation can be provided as a depot preparation, for administration by implantation; e.g., for subcutaneous, intradermal, intra-ocular, periocular or intramuscular injection. Thus, for example, the composition of ETC-216 can be formulated with suitable polymeric or hydrophobic materials (e.g., as an emulsion in an acceptable oil) or ion exchange resins, or as sparingly soluble derivatives; e.g., as a sparingly soluble salt form of the ETC-216.

Targeting of ocular tissues may be accomplished in any one of a variety of ways. Injection into the aqueous or vitreous humor of the eye is one means. Directly injecting the ETC-216 into the proximity of the RPE or Bruch's membrane provides targeting of the complex with some forms of ARMD. In one embodiment, the complex is administered via intra-ocular sustained delivery (such as Vitrasert® or Envision® by Bausch and Lomb). In another embodiment, the compound is delivered by posterior sub-tenons periocular injection.

In certain embodiments, the pharmaceutical formulation of the ETC-216 is a unit dose package. As is known to those of skill in the art, a unit dose package provides delivery of a single dose of a drug to a subject. The compositions and methods of the invention provide a unit dose package of a pharmaceutical formulation comprising, for example, 1050 mg of Apo A-I Milano protein per package. For example, 1050 mg of Apo A-I Milano protein is an amount that administers 15 mg/kg of Apo A-I Milano to a 70 kg subject. The unit can be, for example, a sterile single use vial, a sterile pre-filled syringe, a sterile pre-filled bag (i.e., piggybacks) and the like.

In other embodiments, the pharmaceutical formulation is a unit-of-use package. As is known to those of skill in the art, a unit-of-use package is a convenient, prescription size, patient ready unit labeled for direct distribution by health care providers. A unit-of-use package contains a pharmaceutical formulation in an amount necessary for a typical treatment interval and duration for a given indication. The compositions and methods of the invention provide for a unit-of-use package of a pharmaceutical formulation comprising the ETC-216 in an amount sufficient to treat an average sized adult male or female with, for example, 15 mg/kg of Apo A-I Milano protein intravenously once weekly for 5 weeks. Thus, a unit-of-use package may comprise five doses of the ETC-216 (available in a vial or pre-filled syringes). In one embodiment, the unit-of-use package comprises a pharmaceutical formulation comprising the ETC-216 in an amount sufficient to treat an average sized adult male or female with a dose of 15 mg/kg, 30 mg/kg or 45 mg/kg of Apo A-I Milano once weekly for 5 weeks. It will be apparent to those of skill in the art that the doses described herein are based on the subject's body weight.

The pharmaceutical formulations comprising the ETC-216 must be of a suitable pH, osmolality, tonicity, purity and sterility to allow safe administration to a subject.

The pharmaceutical formulations of ETC-216 are described in U.S. published application US20050142180, published on Jun. 30, 2005, U.S. published application US20040038891, published on Feb. 26, 2004 and U.S. published application US20030109442, published on Jun. 12, 2003, which are hereby incorporated by reference in their entireties.

4.3.5 Use of ETC-216 for Treating Age-Related Macular Degeneration (ARMD)

The doses of ETC-216 encompassed by the invention are described herein. Doses useful for treatment of age-related macular degeneration include doses up to about 50 mg/kg of the ETC-216 administered intravenously to a subject in need thereof. In certain embodiments, the compositions and methods comprise administration of the ETC-216 at a dose of about 1 mg/kg to about 50 mg/kg of a subject's body weight. In particular embodiments, the compositions and methods comprise intravenous administration of ETC-216 at a dose of about 10 mg/kg to about 50 mg/kg. In other embodiments, the composition and methods comprise intra-ocular administration of ETC-216 at a dose of about 0.5 to about 2.5 mg/kg. In other embodiment, the compositions and methods comprise intravenous administration of a pharmaceutical formulation of the ETC-216 at a dose of about 15 mg/kg. In another embodiment, the compositions and methods comprise administration of the ETC-216 at a dose=of about 30 mg/kg. In yet another embodiment, the compositions and methods comprise administration of the ETC-216 at a dose of about 45 mg/kg. The compositions and methods can also comprise the use of a 15 mg/kg dose or 30 mg/kg dose either alone or in combination with the 45 mg/kg dose for the treatment of age-related macular degeneration or related disorders.

It is understood by those of skill in the art that the actual dose of ETC-216 according to the present invention can vary with the route of administration, height, weight, age, severity of illness of the subject, the presence of concomitant medical conditions and the like. For example, an elderly subject with compromised renal or liver function can be treated with a dose of the ETC-216 that is at the lower range of about 1 mg/kg dose (e.g., 0.8 mg/kg or 0.9 mg/kg) as part of the therapy. A subject with severe cardiovascular disease or related disorders that is obese with good renal and liver function can be treated with a dose of the ETC-216 that is, for example, at the upper range of about 45 mg/kg dose (e.g., 50 mg/kg, 48 mg/kg, 45 mg/kg and the like) as part of the therapy. These doses achieve a range of circulating concentrations that include the effective dose with an acceptable risk-to-benefit profile.

The dose of ETC-216 can vary over the duration of treatment. For example, a subject can be treated with the dose of 45 mg/kg of the pharmaceutical formulation of the ETC-216, intravenously once weekly for 3 weeks and then treated with 15 mg/kg of the ETC-216 once every four months or once per year for the lifetime of the subject. Such intermittent doses can be administered to maintain a reduced size and number of drusens in Bruch's membrane. Intermittent doses during the lifetime of the subject to maintain a reduced volume of lipids in the drusens are within the scope of the present invention.

In certain embodiments, a single high dose (e.g., 50 mg/kg or 45 mg/kg) of the ETC-216 is administered intravenously to the subject. In other embodiments, one or more high doses of the ETC-216 are administered to the subject followed by one or more of the same or lower doses of the ETC-216 (e.g., about 1, 5, 10, 15, 45, or 50 mg/kg). Additionally, the opposite regimen may be used comprising administration of one or more low doses of the ETC-216 (e.g., about 1, 5, 10 or 15 mg/kg) followed by one or more of the same or higher doses of the ETC-216 (e.g., about 1, 5, 10, 15, 45 or 50 mg/kg). For more advanced stages of macular degeneration (e.g., wet forms), the high dose in certain embodiments is delivered first.

In certain embodiments, the ETC-216 is administered as an intravenous infusion. In certain embodiments, the compositions and methods comprise administration as a intravenous push infusion. In certain embodiments, the ETC-216 is administered intravenously by intravenous push infusion over a short time period, such as up to 5 minutes, for example, 2-5 minutes. In certain embodiments, administration of the ETC-216 comprises a continuous intravenous infusion. In certain embodiments, ETC-216 is administered by continuous intravenous infusion over a period of time, for example, about 1 hour to 3 hours, preferably, about 30 minutes to 120 minutes. Continuous intravenous infusions can be administered with the aid of an infusion pump or device. In certain embodiments, administration of the ETC-216 can be a combination of continuous intravenous infusions and intravenous push infusions (“bolus doses”). The bolus doses can be administered before, after or during the continuous infusion.

The compositions and methods provide for intravenous infusion of the Apo A-I Milano component of the regimen. Any suitable blood vessel can be used for infusion, including peripheral vessels, such as the vessels in the antecubital fossa of the arm or a central line into the major veins of the chest. In certain embodiments, the pharmaceutical formulation is infused into the cephalic or median cubital vessel at the antecubital fossa in the arm of a subject.

4.3.6 Timing of Adminstration of ETC-216

In certain embodiments, the methods for the treatment of age-related macular degeneration or related conditions comprise administering the ETC-216 about every week, about every other week, about every 5, 6, 7, 8 to 10 or 11 to 14 days to a subject in need thereof. In one embodiment, the ETC-216 can be administered about every 7 days. In certain embodiments, administration of the ETC-216 can be a one-time administration every six months or every year. In certain embodiments, administration can continue for about 1 week, about 2 weeks, about 3 weeks, about 4 weeks, about 5 weeks, about 6 weeks, about 7-12 weeks, about 13-24 weeks or about 25-52 weeks. In certain embodiments, administration of the ETC-216 is about every 7 days for about 5 weeks. In certain embodiments, administration can be intermittent after, for example, about 5 weeks. For example, a subject can be treated once a week for about 5 weeks and then treated about 3 to about 4 times over the following year. In certain embodiments, the pharmaceutical formulations described herein can be administered to the subject intermittently to maintain a reduced size and number of drusens in Bruch's membrane. For example, the ETC-216 in a dose of about 15 mg/kg can be administered about every 10 days for about 7 weeks and then administered, for example, about 26 weeks later or about 52 weeks later.

4.4 Patients and Diseases to be Treated by the Therapy

The invention provides novel compositions and methods to treat age-related macular degeneration or related disorders or forms (wet, dry, early, late).

In some embodiments, the compositions and methods of the present invention are to treat age-related macular degeneration or related disorders in subjects with signs or symptoms thereof. Age-related macular degeneration (ARMD) is a clinical diagnosis based upon the presence of visual disturbance and characteristic findings on dilated eye examination. In patients with dry type of ARMD, drusens are visible on dilated eye examination. In such patients, round or oval patches of geographic atrophy of the retina may be evident as areas of depigmentation, increased pigmentation may be seen with RPE pigmentary modeling. In patients with wet type of ARMD, dilated examination may reveal subretinal fluid, hemorrhage, and lipid exudates. In such patients, neovascularization appears as a grayish discoloration in the macular area. Additional information on age-related macular degeneration (ARMD), including epidemiology, etiology/risk factors, clinical presentation, diagnosis, treatment and prevention is included in Jorge G. Arroyo, “Age-related macular degeneration-I and II,” UpToDate (on-line service) (last modified Sep. 19, 2005), which is hereby incorporated herein by reference.

There are two possible explanations for RPE and retina ischemia. One is decreasing hydraulic conductivity through Bruch's membrane and the other is decreased choroidal perfusion. Choroidal perfusion normally decreases with age and decreases more severely in patient with ARMD. Worsening levels of choroidal perfusion are accosiated with more severe levels of ARMD (Spraul et al., Invest Ophtalmol Vis Sci, 39(11):2201-2202, 1998; Grunwald et al., Invest Ophtalmol Vis Sci, 46(3):1033-1038, 2005; Ciulla et al., Br J Ophtalmol. 86(2):209-213, 2002). Also, there is a histologic evidence of choroidal arteriosclerosis (Curcio et al., Invest Ophtalmol Vis Sci, 42:265, 2001). Without being bound in theory, it is proposed that if Apo A-I Milano works to promote overall reduction in arteriosclerosis, one can expect choroidal arteriosclerosis to lessen and choroidal perfusion to improve. Improved choroidal perfusion would, in turn, lessen RPE and retinal ischemia thus helping reverse ARMD.

In one embodiment, the compositions and methods of the invention provide for treating age-related macular degeneration or related disorders in subjects at risk for developing ARMD. A number of possible risk factors have been identified, of which age, smoking and family histort are some of the strongest factors that appear to definitively increase risk of ARMD. Additional risk factors are set forth in Jorge G. Arroyo, “Age-related macular degeneration-I and II,” UpToDate (on-line service), as referenced above.

### 5. EXAMPLES

The present invention will be further understood by reference to the following non-limiting examples.

### 5.1. Example 1

### Formulation of ETC-216

ETC-216 is a recombinant apolipoprotein A-I Milano/1 palmitoyl-2-oleoyl phosphatidylcholine complex in a 1 to 0.9 ratio by weight. Stock solutions of ETC-216 contain 14.5 mg protein/ml in a sucrose mannitol buffer.

### 5.2. Example 2

### Comparative Study of the Effect of Apo A-I Milano Mutation on the Development of ARMD in Apo A-I Milano Mutation Carriers

The aim of this study was to determine the effect of Apo A-I Milano mutation on the development of ARMD in Apo A-I Milano mutation carriers.

The study was carried out in Limon sul Garda, Italy. The study group was presented by family with Apo A-I Milano mutation and consisted of group of 7 people of >50 years old. The control group consisted of group of 24 people of >50 years old. Both groups consisted of smokers and non-smokers.

The parameters determined to be indicative for the development of ARMD were: drusens >63 microns, any size of drusens combined with hyperpigmentation and any size of drusens combined with hypopigmentation.

The results are presented in tables 1 and 2.

TABLE 1
Effect of Apo A-I Milano mutation on number of drusens

| | | Drusen >63 | Drusen >125 |
|---|---|---|---|
| | Drusen | microns | microns |
| Apo A-I Milano (n = 7) | 2.11 | 1.46 | 0 |
| Controls (n = 24) | 11.36 | 7.08 | 1.89 |
| P-value (T test) | 0.29 | 0.30 | 0.35 |

TABLE 2
Effect of Apo A-I Milano mutation on hyperpigment RPE and hypopigment RPE

| | Hyperpigment RPE | | Hypopigment RPE | | ARMD | |
|---|---|---|---|---|---|---|
| Apo A-I Milano | 0/14 | eyes (0%) | 5/14 | eyes (35%) | 5/14 | eyes (36%) |
| (n = 7) | | | | | | |
| Controls (n = 24) | 18/48 | eyes (37.5%) | 29/48 | eyes (60%) | 30/48 | eyes (63%) |

| | | | |
|---|---|---|---|
| P-value (T test) | 0.006 | 0.21 | 0.13 |

The study demonstrated that the Apo A-I Milano mutation carriers have fewer drusens, fewer drusens >63 microns and fewer drusens >125 microns. The RPE hyperpigmenation and hypopigmentation was significantly less evident in the study group. Overall, the Apo A-I Milano mutation carriers demonstrated less ARMD in comparison with the control group.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below. All references cited herein are hereby incorporated by reference in their entireties.

What is claimed:

1. A method of treating or preventing age-related macular degeneration in a subject in need thereof comprising administering to the subject an Apolipoprotein A-I Milano (Apo A-I Milano or AIM) or a variant thereof.

2. The method of claim 1, wherein Apo A-I Milano or the variant is administered as a protein lipid complex.

3. The method of claim 2, wherein said complex is administered at a intravenous dose of about 1 mg/kg to about 50 mg/kg.

4. The method of claim 2, wherein said complex is administered at an intra-ocular dose of about 0.1 to about 3 mg/kg.

5. The method of claim 2, wherein said complex is administered at a dose of about 10 mg/kg to about 45 mg/kg.

6. The method of claim 2, wherein said complex is administered at a dose of about 15 mg/kg to about 45 mg/kg.

7. The method of claim 2, wherein said complex is administered at a dose of about 10 mg/kg.

8. The method of claim 2, wherein said complex is administered at a dose of about 15 mg/kg.

9. The method of claim 2, wherein said complex is administered at a dose of about 45 mg/kg.

10. The method of claim 2, wherein said complex is administered at a dose of about 30 mg/kg.

11. The method of any one of claims 2-10, wherein the protein lipid complex is an Apolipoprotein A-I Milano: 1-palmitoyl-2-oleoyl-sn-glycero-3-phosphocholine complex (Apo A-IM: POPC complex).

12. The method of any one of claims 1-11, wherein the age-related macular degeneration is nonexudative or dry age-related macular degeneration.

13. The method of any one of claims 1-11, wherein the age-related macular degeneration is exudative or wet age-related macular degeneration.

14. The method of any one of claims 1-13, wherein the Apo A-I Milano is a recombinant Apo A-I Milano.

15. The method of any one of claims 2-14, wherein the lipid is a phospholipid, cholesterol, triglyceride, or cholesterol ester.

16. The method of claim 14, wherein the lipid is a phospholipid.

17. The method of claim 16, wherein the phospholipid is selected from the group consisting of phosphatidylcholine, egg phosphatidylcholine, soybean phosphatidylcholine, dipalmitoylphosphatidylcholine, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, brain sphingomyelin, dipalmitoylsphingomyelin and distearoylsphingomyelin.

18. The method of claim 17, wherein the phospholipid is a 1-palmitoyl-2-oleoylphosphatidylcholine (POPC).

19. The method of claim 11, wherein the AIM:POPC complex has a ratio of about 1:0.8 to about 1:1.0 by weight.

20. The method of claim 19, wherein the AIM:POPC complex has a ratio of about 1:0.9 by weight.

21. The method of claim 20, wherein the complex is administered as a pharmaceutical formulation.

22. The method of claim 21, wherein the pharmaceutical formulation comprises a buffer comprising sucrose and mannitol, and wherein said Apo A-I Milano is recombinant Apo A-I Milano at a concentration of about 12 mg per ml to about 18 mg per ml of buffer.

23. The method of claim 22, wherein the concentration of the recombinant Apo A-I Milano is 14.5 mg per ml of buffer.

24. The method of claim 23, wherein the pharmaceutical formulation comprises an aqueous buffer comprising glucose and sodium phosphate, and wherein said buffer has a pH of about 7.0 to 7.8.

25. The method of claim 23 or 24, wherein said buffer is 2% glucose and 4 mM of sodium phosphate, and wherein said pH is about 7.4.

26. The method of any one of claims 21-25, wherein the pharmaceutical formulation is a sterile liquid pharmaceutical formulation.

27. A method of treating or preventing age-related macular degeneration in a subject in need thereof, comprising administering to the subject an Apolipoprotein A-I Milano (Apo A-I Milano or AIM) in an amount sufficient to promote reverse cholesterol transport in the subject such that age-related macular degeneration is treated.

28. A pharmaceutical composition comprising an Apolipoprotein A-I Milano: 1-palmitoyl-2-oleoyl-sn-glycero-3-phosphocholine complex (Apo A-IM: POPC complex) and a pharmaceutically acceptable carrier for treatment or prevention of age-related macular degeneration.
